SEQUENCE LISTING (2 sequences)

SEQ ID NO: 1 - DNA, 980 bp, Mus musculus
  CDS (87)..(656)
caagcgcgcaagagagcgggctgcctcgcagtccgagccggagagagggagcgcgacgcgcgcggccccggacgccctccgaaaccatgaactttctgctctcttgggtgcactggaccctggctttactgctgtacctccaccatgccaagtggtcccaggctgcacccacgacagaaggagagcagaagtcccatgaagtgatcaagttcatggacgtctaccagcgaagctactgccgtccaattgagaccctggtggacatcttccaggagtaccccgacgagatagagtacatcttcaagccgtcctgtgtgccgctgatgcgctgtgcaggctgctgtaacgatgaagccctggagtgcgtgcccacgtcagagagcaacatcaccatgcagatcatgcggatcaaacctcaccaaagccagcacatagagagaatgagcttcctacagcacagcagatgtgaatgcagaccaaagaaagacagaacaaagccagaaaatcactgtgagccttgttcagagcggagaaagcatttgtttgtccaagatccgcagacgtgtaaatgttcctgcaaaaacacagactcgcgttgcaaggcgaggcagcttgagttaaacgaacgtacttgcagatgtgacaagccaaggcggtgagccaggctggcaggaaggagcctcctcagggtttcgggaaccagacctctcaccggaaagaccgattaaccatgtcaccaccatgccatcatcgtcaccgttgacagaacagtccttaatccagaaagcctgatatgaaggaagaggagatccttcgaggagcactttgggtccggagggcgagactccggcagacgcattcccgggcaggtgaccaagcacgtgcctcgtgggactggattcgccattttcttatatctgctgctaaatcgccaagcccggaagattagggttgtttctgggattcctgtagagctcgtg

SEQ ID NO: 2 - protein, 190 aa, Mus musculus
MNFLLSWVHWTLALLLYLHHAKWSQAAPTTEGEQKSHEVIKFMDVYQRSYCRPIETLVDIFQEYPDEIEYIFKPSCVPLMRCAGCCNDEALECVPTSESNITMQIMRIKPHQSQHIERMSFLQHSRCECRPKKDRTKPENHCEPCSERRKHLFVQDPQTCKCSCKNTDSRCKARQLELNERTCRCDKPRR

(19) United States
(12) Patent Application Publication
(10) Pub. No.: US 2005/0220773 A1
(43) Pub. Date: Oct. 6, 2005

(54) Implant containing cells having growhfactor gene transferred thereinto

(76) Inventors: Toshimasa Uemura, Ibaraki (JP); Tetsuya Tateishi, Ibaraki (JP); Kazuya Matsumoto, Shiga (JP); Hiroko Kojima, Ibaraki (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(21) Appl. No.: 10/504,969

(22) PCT Filed: Oct. 21, 2002
(86) PCT No.: PCT/JP02/10866

§ 371 Date: Apr. 6, 2005

(30) Foreign Application Priority Data
Feb. 19, 2002 (JP) 2002-41604

(51) Int. Cl. A61K 48/00; C12N 5/08

(52) U.S. Cl. 424/93.21; 435/366; 424/423

(57) ABSTRACT

This invention provides alternative bone implants that have high bioadaptability and enable rapid bone regeneration. Specifically, this invention relates to implants consisting of a bioadaptable material comprising cells transfected with growth factor genes. Such implants are produced by inoculating bone-marrow-derived cells transfected with the genes of vascular endothelial growth factors (VEGF) into a bioadaptable material, such as porous ceramics, and culturing them.

### TECHNICAL FIELD

The present invention relates to implants comprising cells transfected with growth factor genes and a process for producing the same. More particularly, the present invention relates to alternative bone implants that enable rapid bone regeneration with the aid of overexpressed vascular endothelial growth factors.

### BACKGROUND ART

Up to the present, tissues with a limited capacity for regeneration, such as bone, have been regenerated by transplanting autogenous tissues or complementing or replacing tissues with artificial implants. Use of a patient's autogenous tissues, however, imposes a heavy burden on a patient, and the amounts thereof that can be obtained are limited. Also, mechanical and structural properties or bioadaptability as good as those of natural tissues cannot be expected from artificial implants.

Meanwhile, research regarding “regenerative medicine” has made progress. In regenerative medicine, cells harvested from a body are cultured in vitro and organized to reconstruct tissues that are as similar as possible to those in the body. The resulting tissues are then returned to the body. If such regenerative medicine comes to pass, it would be the most ideal therapy for repairing tissue defects. In the field of regenerative medicine, in vitro tissue regeneration is generally carried out by inoculating cells into an adequate scaffold and culturing them. There are several critical issues in regenerative medicine. For example, cells need to be proliferated and differentiated to tissues of interest as rapid as possible at in vitro cell culture. Also, the transplanted tissues need to quickly proliferate, fuse in the defects, and be reconstructed after implantation into the body.

In order to deal with such issues, several techniques in which cytokines (humoral factors) responsible for cell differentiation are directly introduced to cells are known. For example, JP Patent Publication (Kokai) No. 2001-316285 A discloses a technique of culturing bone marrow cells in a collagen sponge adsorbed with TGF-β1. JP Patent Publication (Kokai) No. 8-3199 A (1996) discloses a material for regenerating cartilage tissues using a collagen-cartilage cell composite comprising bFGF. In these techniques, however, the growth factors are directly added to cells, and activity of the growth factors cannot be maintained for a sufficiently long time. Because, the growth factors added are rapidly diffused in the body, and the effect thereof is drastically lowered within several hours to 1 day.

In the case of regeneration of tissues such as those of the liver, angiogenesis is known to be a critical process thereof (Ajioka, I. et al., Hepatology 29, pp. 396-402, 1999). However, the effect of angiogenesis on bone regeneration has not yet been fully examined.

### DISCLOSURE OF THE INVENTION

An object of the present invention is to provide alternative bone implants that have high bioadaptability and enable rapid bone regeneration.

The present inventors have conducted concentrated studies in order to attain the above object. As a result, they have come to consider that the effects of the growth factors could be maintained and tissues are more rapidly regenerated by transfecting the growth factor genes into the cells and allowing them to overexpress therein. They have also found that transfection of vascular endothelial growth factor (VEGF) gene that accelerated angiogenesis in the cells can remarkably improve bone regeneration. This has led to the completion of the present invention.

Specifically, the present invention relates to the following (1) to (12).

(1) Alternative bone implants consisting of a bioadaptable material that comprises cells transfected with growth factor genes.

(2) The implants according to (1), wherein the growth factors accelerate angiogenesis and/or osteogenesis.

(3) The implants according to (2), wherein the growth factor is a vascular endothelial growth factor (VEGF).

(4) The implants according to any one of (1) to (3), wherein the cells are embryonic stem cells or bone-marrow-derived mesenchymal stem cells.

(5) The implants according to (4), wherein the cells are osteoblasts.

(6) The implants according to any one of (1) to (5), wherein the cells are harvested from a patient.

(7) The implants according to any one of (1) to (6), wherein the bioadaptable material is selected from the group consisting of hydroxyapatite, α-TCP, β-TCP, collagen, polylactic acid, polyglycolic acid, and a complex of two or more thereof.

(8) A method for producing alternative bone implants comprising the following procedures of:
1) inducing differentiation of bone-marrow-derived cells into osteoblasts in vitro;
2) transfecting growth factor genes into the cells; and
3) inoculating the cells into bioadaptable material for proliferation.

(9) The method according to (8), wherein the growth factor is a vascular endothelial growth factor (VEGF).

(10) The method according to (8) or (9), wherein the bioadaptable material is selected from the group consisting of hydroxyapatite, α-TCP, β-TCP, collagen, polylactic acid, polyglycolic acid, and a complex of two or more thereof.

(11) The method according to any one of (8) to (10), wherein the growth factor genes are transfected using an adenoviral or retroviral vector.

(12) The method according to any one of (8) to (11), wherein differentiation is induced with the aid of a member selected from the group consisting of dexamethasone, immunosuppressants, bone morphogenetic proteins, and osteogenic humoral factors.

Hereafter, the present invention is described in detail.

1. Constitution of Implants

The implants according to the present invention are alternative bone implants consisting of a bioadaptable material that comprises cells transfected with growth factor genes.

1.1 Growth Factors

The growth factors that are employed in the implants according to the present invention are not particularly limited. Examples thereof include basic fibroblast growth factor (bFGF), platelet-derived growth factor (PDGF), insulin and insulin-like growth factor (IGF), hepatocyte growth factor, (HGF), glial-derived neurotrophic factor (GDNF), neurotrophic factor (NF), hormones, cytokines, bone morphogenetic protein (BMP), transforming growth factor (TGF), and vascular endothelial growth factor (VEGF).

Growth factors that accelerate angiogenesis and/or osteogenesis are particularly preferable. Examples thereof include bone morphogenetic protein (BMP), bone growth factor (BGF), vascular endothelial growth factor (VEGF), and transforming growth factor (TGF). Among them, vascular endothelial growth factor (VEGF) is the most preferable since it remarkably improves angiogenesis in vitro and it enables rapid bone regeneration.

The growth factor genes can be prepared by conventional techniques, using their known sequences. For example, cDNA of a target growth factor gene can be prepared by isolating RNA from the osteoblast, preparing primers based on a known sequence of the growth factor gene, and cloning the cDNA by PCR using the RNA and the primers. Alternatively, genes may be purchased or provided by others.

1.2 Cells

The cells that are employed in the present invention are undifferentiated cells with differentiation and proliferation ability. Examples thereof include the mesenchymal stem cells, the hematopoietic stem cells, the skeletal muscle stem cells, the neural stem cells, and the hepatic stem cells. ES (embryonic stem) cells and bone-marrow-derived mesenchymal stem cells are particularly preferable.

In addition to established cell lines, cells harvested from the body of a patient may also be used. These cells are preferably prepared by removing connective tissues and the like in accordance with a conventional technique after being harvested from the body of the patient. Alternatively, cells may be primarily cultured to grow before use in accordance with a conventional technique.

1.3 Bioadaptable Material

The bioadaptable material that is employed in the present invention can be used as a scaffold for cell culture. At the same time, it is transplanted into the body together with cells and it works as an alternative bone implant. The term “bioadaptable material” used herein refers to a material that is highly compatible with the body and highly safe. Examples thereof include: metallic materials such as SUS316L, vitallium, and Ti-6A1-4V; polymeric materials such as ultra-high molecular weight polyethylene, MMA bone cement, polylactic acid, polyglycolic acid, polyethylene terephthalate, and polypropylene; and ceramic materials such as hydroxyapatite, β-TCP, α-TCP, and bioglass. Use of porous ceramic materials, such as hydroxyapatite, β-TCP, or α-TCP, collagen, polylactic acid, polyglycolic acid, a composite thereof, or synthetic absorbable polymers is preferable for the application thereof as a scaffold for cell culture.

The bioadaptable materials are preferably porous for uniform inoculation of cells. In the present description, the term “porous” refers to a porosity of 40% or higher. The pore size is not particularly limited, although a diameter of 200 μm to 500 μm is preferable for easy bone regeneration.

The most suitable bioadaptable material may be determined depending on a purpose and site for implants. For example, hydroxyapatite is preferable for an implantation site (or a surgical technique) that requires mechanical strength. In contrast, biodegradable β-TCP or the like is preferable for an implantation site (or a surgical technique) that does not require mechanical strength.

The configurations and shapes of the bioadaptable materials are not particularly limited. Bioadaptable materials can take any desired configurations or shapes, such as sponges, meshes, unwoven fabric products, discs, films, sticks, particles, or pastes. These configurations and shapes may be suitably selected depending on their applications.

2. Method for Preparing Implants

The implants according to the present invention are produced via the following procedures:
1) inducing differentiation of human bone-marrow-derived cells into osteoblasts in vitro;
2) transfecting growth factor genes into the cells; and
3) inoculating the cells into bioadaptable material for proliferation.

Each procedure is hereafter described in detail.

2.1 Induction of Cell Differentiation

The cells should be induced to differentiate into cells for constructing target tissues using an appropriate agent. For example, at least one member selected from the group consisting of dexamethasone, immunosuppressants agents such as FK-506 and cyclosporine, bone morphogenetic proteins (BMPs) such as BMP-2, BMP-4, BMP-5, BMP-6, BMP-7, and BMP-9, and osteogenic humoral factors such as TGFβ is supplemented to the medium to induce cell differentiation into bone cells.

2.2 Transfection of Growth Factor Genes

The growth factor genes can be prepared by conventional techniques, using their known sequences. For example, cDNA of a target growth factor gene can be prepared by isolating RNA from the osteoblast, preparing primers based on a known sequence of the growth factor gene, and cloning the cDNA by PCR using the RNA and the primers.

In the present invention, the growth factor genes can be transfected into the target cell by a common method for transfection into animal cells. Examples of the methods that can be employed include the calcium phosphate method, lipofection, electroporation, microinjection, and a method using a retroviral or baculoviral vector. From the viewpoints of safety and transfection efficiency, a method using an adenoviral or retroviral vector is preferable, and a method using an adenoviral vector is most preferable.

The adenoviral vector can be constructed according to a known technique, such as the method of Miyake et al. (Miyake, S. et al., Proc. Natl. Acad. Sci. 93: 1320-1324, 1993). Alternatively, a commercially available kit, such as the Adenovirus Cre/loxP Kit (Takara Shuzo Co., Ltd.), can be used. This kit is used for constructing a recombinant adenoviral vector utilizing a novel regulation system of gene expression (Kanegae Y. et al., 1995, Nucl. Acids Res. 23, 3816) that employs Cre recombinase of P1 phage and its recognition sequence, loxP. This kit can be used to simply construct a recombinant adenoviral vector carrying the transcription factor gene.

The multiplicity of infection (moi) of adenovirus infection is 10 or more, preferably 50 to 200, and more preferably around 100 (approximately 80 to 120).

2.3 Cell Culture

Cells transfected with the aforementioned growth factor genes may be cultured in accordance with a conventional technique by inoculating the cells into a scaffold consisting of the aforementioned bioadaptable material.

Cells may be simply inoculated into the bioadaptable scaffold or inoculated in the form of mixtures with a liquid such as a buffer, physiological saline, a solvent for injection, or a collagen solution. When the cells do not smoothly enter into a pore because of its porous structure of the material, cells may be inoculated under low pressure.

Preferably, the number of cells to be inoculated (inoculation density) is adequately determined in accordance with the types of cells or scaffolds in order to regenerate tissues more efficiently while maintaining the morphology of the cells. For example, the inoculation density is preferably 1,000,000 cells/ml or higher in the case of osteoblasts.

Cell culture is carried out in the presence of a bioadaptable scaffold. A conventional medium for cell culture, such as MEM medium, α-MEM medium, or DMEM medium, can be suitably selected depending on the type of cell to be cultured. FBS (Sigma), antibiotics such as Antibiotic-Antimycotic (GIBCO BRL), and other substances may be added to the medium. Culture is preferably conducted in the presence of 3% to 10% CO₂ at 30° C. to 40° C., and particularly preferably in the presence of 5% CO₂ at 37° C. The culture period is not particularly limited, and it is at least 4 days, preferably at least 7 days, and more preferably at least 2 weeks.

3. Application of Implants

The tissues regenerated by the aforementioned method are transplanted or injected into a body together with the bioadaptable scaffolds. Thus, they can be used as alternative bone implants.

The configurations and shapes of the implants of the present invention are not particularly limited. Implants can take any desired configurations or shapes, such as sponges, meshes, unwoven fabric products, discs, films, sticks, particles, or pastes. These configurations and shapes may be suitably selected depending on their applications.

The implants according to the present invention may suitably comprise other components within the scope of the present invention. Examples of such components include: growth factors such as basic fibroblast growth factor (bFGF), platelet-derived growth factor (PDGF), insulin and insulin-like growth factor (IGF), hepatocyte growth factor (HGF), glial-derived neurotrophic factor (GDNF), neurotrophic factor (NF), hormones, cytokines, bone morphogenetic protein (BMP), transforming growth factor (TGF), and vascular endothelial growth factor (VEGF); inorganic salts such as St, Mg, Ca, and CO₃; organic substances such as citric acid and phospholipid; and drugs.

The implants according to the present invention comprise bone cells and tissues reconstructed from the cells to which the growth factor genes have been transfected. Bone cells and tissues may be reconstructed not only prior to the application of the implants (in vitro) but also after application of the implants into bone defects (in vivo). The implants according to the present invention have high compatibility with bones and high ability for osteogenesis. Thus, they can be integrated with natural bones and can replace bone defects immediately after implantation into a body.

### BRIEF DESCRIPTION OF THE DRAWINGS

FIG. 1 is an image showing the results of X-gal staining of the uninfected cells (the control) and the adenovirus-infected cells (AD-lacZ).

FIG. 2 is a graph showing the efficiency of transfecting the LacZ genes into the adenovirus-infected cells (evaluated based on the staining intensity).

FIG. 3 shows the results of Northern hybridization representing changes in the expression levels of VEGF affected by the multiplicity of infection (moi).

FIG. 4 is a graph showing the changes in the expression levels of VEGF (relative to the expression level of 18s rRNA) affected by moi.

FIG. 5 shows the results of Northern hybridization representing changes in the expression levels of VEGF with the elapse of time.

FIG. 6 is a graph showing the changes in the expression levels of VEGF (relative to the expression level of 18s rRNA) with the elapse of time.

FIG. 7 is a graph showing the effect of moi on the VEGF levels in the culture medium (A: 4th day; B: 7th day).

FIG. 8 is a graph showing the changes in the expression levels of VEGF infected at a moi of 100 with the elapse of time.

FIG. 9 is a graph showing the growth curve of the human umbilical vein endothelial cells (HUVEC) affected by VEGF.

FIG. 10 is a photograph showing the results of observing the regeneration of bone tissues via hematoxylin-eosin staining (A: the control (10 days), B: the control (20 days), C: VEGF (10 days), and D: VEGF (20 days)).

This description includes part or all of the contents as disclosed in the description of Japanese Patent Application No. 2002-41604, which is a priority document of the present application.

### BEST MODES FOR CARRYING OUT THE INVENTION

The present invention is hereafter described in greater detail with reference to the examples, although the technical scope of the present invention is not limited thereto.

### EXAMPLE 1

### Acceleration of Angiogenesis by VEGF Gene-Transfected Rat Osteoblasts

1. Experimental Method

1) Preparation of Adenoviral Vector

(i) cDNA of Mouse VEGF

cDNA (SEQ ID NO: 1) of mouse VEGF was provided by Dr. Watanabe of the Tokyo Institute of Technology.

(ii) Preparation of Recombinant Adenoviral Vector

The cDNA of VEGF was inserted into the SwaI site of the cosmid vector pAxCAwt using the commercialized Adenovirus Cre/loxP Kit (Takara Shuzo Co., Ltd.), and a recombinant adenoviral vector was prepared according to the manufacturer's instructions of the kit. The insertion of VEGF was confirmed based on the pattern and the sequence of the restriction enzyme. Since the vector is E1-deleted adenovirus vector, the virus can not replicate in the target cell after infection. Also, this virus vector comprises a stuffer in the upstream region of the target gene and thus expresses the gene only when it is cotransfected with the Cre recombinase-expressing virus. The titer of the prepared virus was approximately 2.4×10⁹ PFU/ml, and infection efficiency was very high.

2) Sampling and Culture of Bone Marrow Cells

The rat bone marrow osteoblasts (RBMO) were obtained from the femur of a 6-week-old Fisher rat (male) in accordance with the method of Maniatopoulos et al. (Maniatopoulos, C., Sodek, J., and Melcher, A. H., 1988, Cell Tissue Res. 254, 317-330). The cells were cultured in MEM medium (Nacalai Tesque) containing 15% FBS (Sigma) and Antibiotic-Antimycotic (GIBCO BRL) until they became confluent. Subsequently, the cells were subcultured in φ3.5 cm dishes with aforementioned medium containing 5 nM dexamethasone (Sigma), 10 mM β-glycerophosphate (Sigma), and 50 μg/ml ascorbic acid phosphate (Wako), at the number of cells per dish of approximately 400,000. The subcultured rat osteoblasts (90% confluent) were infected with the LacZ gene-expressing virus (AD-LacZ) and the Cre recombinase-expressing virus (AD-CRE) at a multiplicity of infection (moi) of 100 on the next day.

3) Observation of LacZ Gene-Expressing Cell by X-Gal Staining

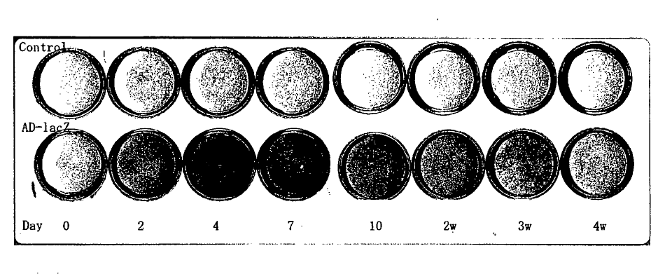

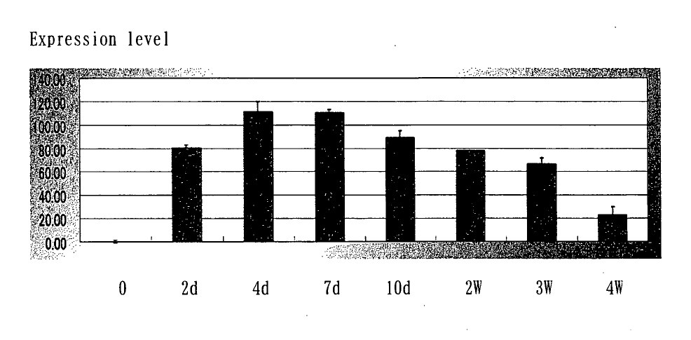

Expression of LacZ in the rat osteoblasts 4 weeks after adenovirus infection was observed by X-gal staining in accordance with the method of Scholer et al. (Scholer, H. R. et al., 1989, EMBO J., 8, 2551-2557) (FIG. 1). The uninfected cells were prepared as the control. The stained cells were subjected to image analysis using the NIH image, and the number of expressed cells was determined to evaluate the efficiency of gene transfection (FIG. 2).

Results: The maximal efficiency of expression was observed 4 days after infection, and the efficiency was 90% or higher. Slight expression was observed until the fourth week.

4) Northern Hybridization (i) [Confirmation of Transcription]

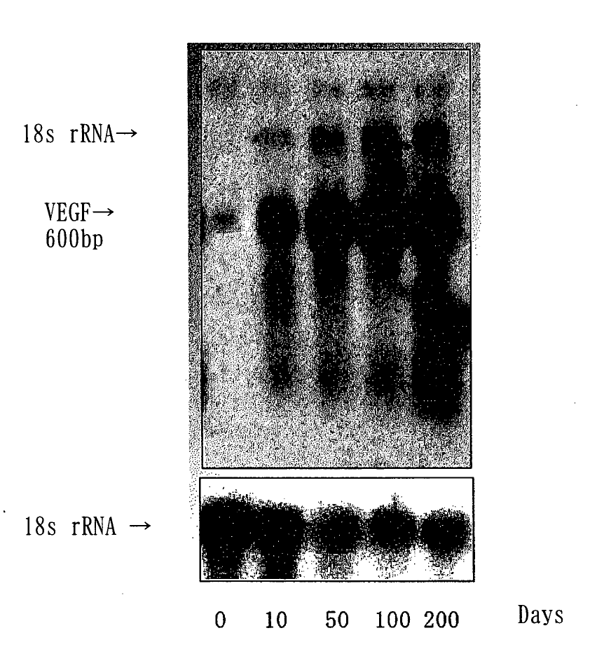

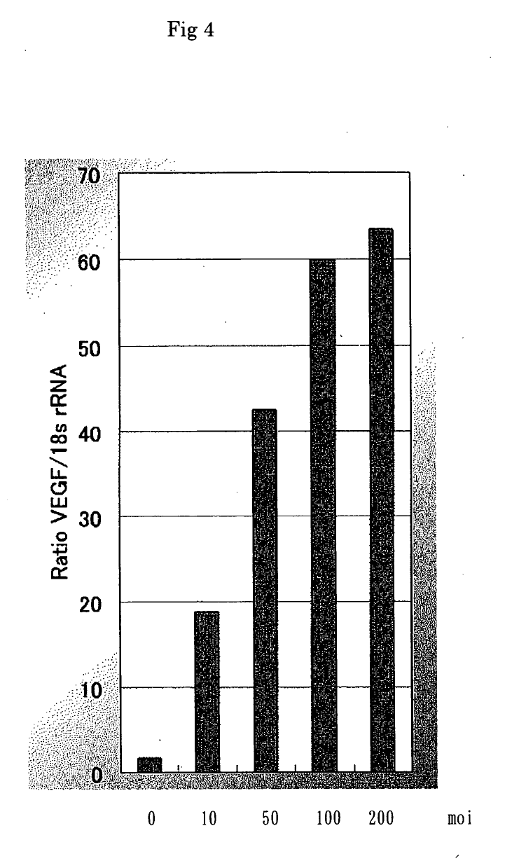

Total RNA was isolated from the rat osteoblasts 1 week after adenovirus infection using a commercialized TRIzol reagent (# 15596-10551, GIBCO BRL) according to the manufacturer's instructions. Total RNA (10 μg) was separated on a 1% agarose/5.5% formaldehyde gel and transferred in 20×SSC to the Hybond™-XL membrane (Amersham Pharmacia Biotech). Thereafter, the membrane was heated at 80° C. for 2 hours and then irradiated with ultraviolet rays for 2 minutes. The cDNA probe of VEGF was labeled with α-³²PdCTP (3,000 Ci/mmol, Amersham Pharmacia Biotech) using Rediprime™ (Amersham Pharmacia Biotech), and the incorporated α-³²PdCTP was removed using a MicroSpin™ G-25 Column (Amersham Pharmacia Biotech). This membrane was incubated in the PerfectHyb™ Plus Hybridization Buffer (Sigma) at 68° C. for 30 minutes, the labeled cDNA probe (2×10⁶ cpm/ml) was added, and incubation was further carried out at 68° C. for 1 hour. The membrane was washed with 2×SSC/0.1% SDS at room temperature for 5 minutes and then washed twice with 0.5×SSC/0.1% SDS at 68° C. (20 minutes each). Thereafter, the membrane was exposed to the Kodak XAR film at −80° C. overnight (FIG. 3). Further, the expression level of VEGF was represented relative to that of 18s rRNA by setting a value attained at an moi of 0 at 1 (FIG. 4).

Results: The expression level of VEGF was found to increase as the degree of moi was elevated.

5) Northern Hybridization (ii) [Changes in VEGF Expression Level with the Elapse of Time]

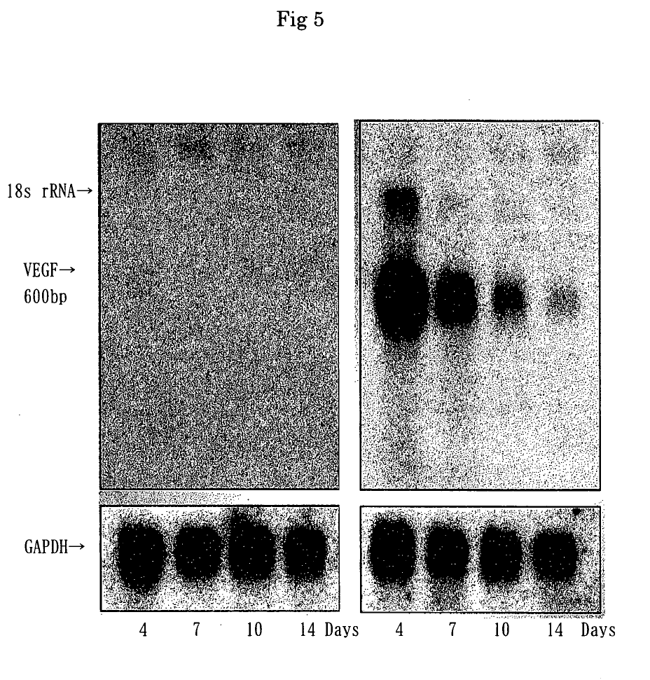

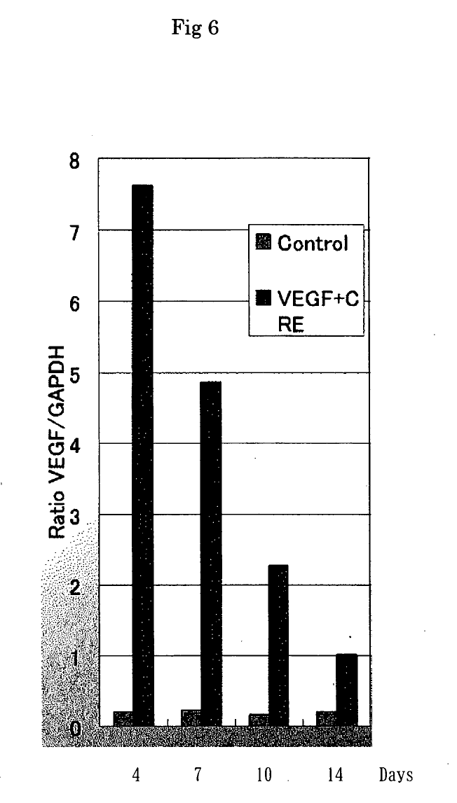

Total RNA was extracted 4 days, 7 days, 10 days, and 14 days after adenovirus infection, and the changes in the expression level of VEGF mRNA were observed by Northern hybridization in the same manner as in the above section. The expression level of VEGF was represented relative to VEGF and GAPDH (FIG. 5, FIG. 6).

6) Confirmation of the VEGF Level in Medium via ELISA (i) [the Effects of moi]

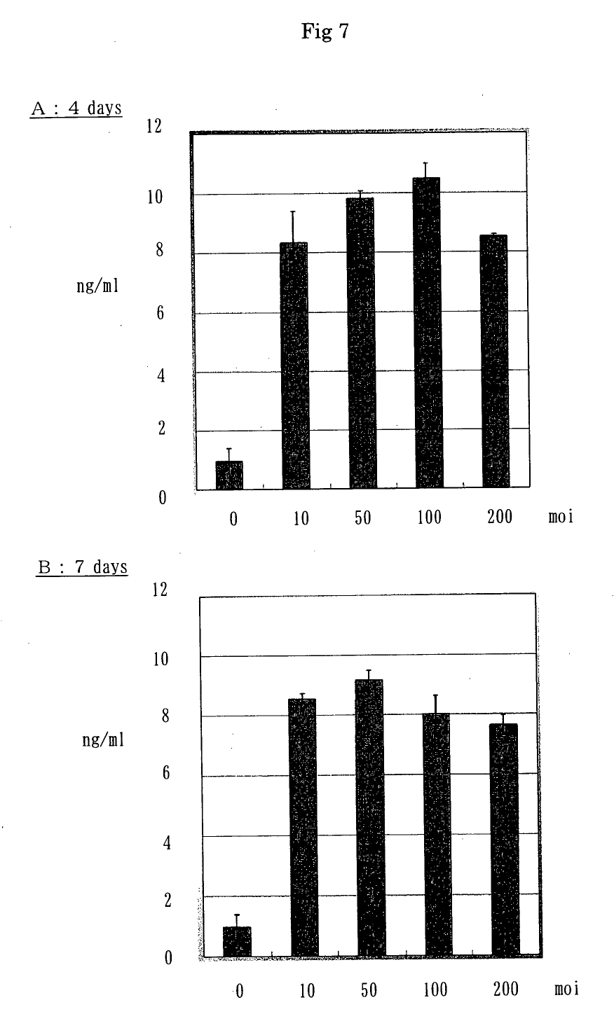

The rat osteoblasts were infected with AD-VEGF at various degrees of moi, and the VEGF level in the medium was assayed via ELISA. The supernatant obtained on the fourth day (FIG. 7-A) and that on the seventh day (FIG. 7-B) were subjected to assay.

Results: Although the expression level was supposed to increase as the degree of moi was elevated, the expression level was maintained at a constant level of approximately 10 ng/ml, regardless of the degree of moi. This phenomenon is considered to have occurred because of a decreased number of cells due to virus infection.

7) Confirmation of VEGF Level in Medium via ELISA (ii) [Changes in the VEGF Level with the Elapse of Time]

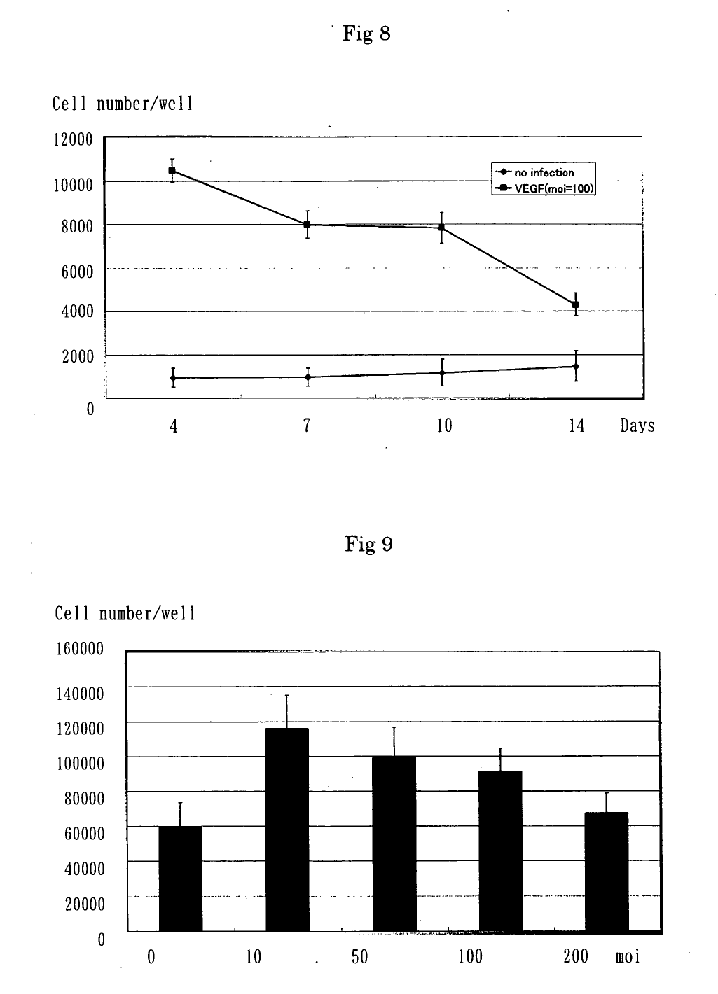

The rat osteoblasts were infected with AD-VEGF at an moi of 100, the VEGF level was assayed via ELISA, and changes thereof were observed with the elapse of time (FIG. 8). Media were exchanged every 3 days, and the supernatant was recovered at that time.

Results: The expression level of VEGF was high up to about the tenth day, and it was significantly lowered on the fourteenth day. This indicates that the effects of the virus for VEGF expression can be maintained for about 2 weeks.

8) Confirmation of Activity of Secreted VEGF Utilizing Human Umbilical Vein Endothelial Cells (HUVEC)

In order to check VEGF activity, human umbilical vein endothelial cells (HUVEC) were seeded onto a 96-well plate, and the supernatant of a medium in which rats osteoblasts infected with viruses at various moi levels had been cultured was added thereto in amounts of 20 μl/well. Thereafter, the growth rate of cells was evaluated using a Cell Counting Kit (Wako) (FIG. 9).

When viral VEGF was present, the cell growth was found to become twice as high as that when no viral VEGF was present.

2. Conclusion

The VEGF expression level in the VEGF-transfected cells was approximately 8 times to 10 times higher than that in uninfected cells, regardless of the degree of moi. An moi of about 50 to 200, and optimally of about 100, was considered to be particularly preferable. Based on a growth experiment in which VEGF-sensitive human umbilical vein endothelial cells (HUVEC) had been used, the growth of the vascular cells was found to be significantly accelerated in cells to which a medium containing AD-VEGF-infected rat bone marrow cells had been added, compared with the case of uninfected cells.

The effects of the growth factors were maintained for about 2 weeks. This was much higher than that in the case where the growth factor itself had been directly transfected (where the growth factors began to diffuse within a period of several hours to 1 day after infection).

### EXAMPLE 2

### Regeneration of Bone Tissues by VEGF Gene-Transfected Rat Osteoblast

1) Testing Method

Bone marrow was obtained from the femur of a Fisher rat and then cultured in a T75 flask containing α-MEM and 15% FBS at 37° C. in the presence of 5% carbon dioxide for 6 days. Thereafter, inducers of differentiation into osteoblasts, such as dexamethasone, β-glycerophosphate, or ascorbic acid, were added, and culture was conducted for an additional 4 days. When the cells became substantially confluent in the T75 flask (1 to 3×10⁷ cells/flask), they were infected with AD-VEGF (moi=100) in the same manner as in Example 1, trypsinized 1 day thereafter, seeded on porous ceramics (Osferion®, Olympus Optical Co., Ltd.; average pore diameter: 200 μm; porosity of 75%) at adensity of 2,000,000 cells/ml or higher, and then cultured under the same conditions as above.

Bone defects were provided on the femur of the Fisher rat 1 day thereafter, and the aforementioned ceramics (2×2×2 mm 3) were implanted therein. The femur was taken out from the rat 2 weeks after implantation and then fixed. Thereafter, sections were prepared and subjected to hematoxylin-eosin staining to observe osteogenesis.

2) Results

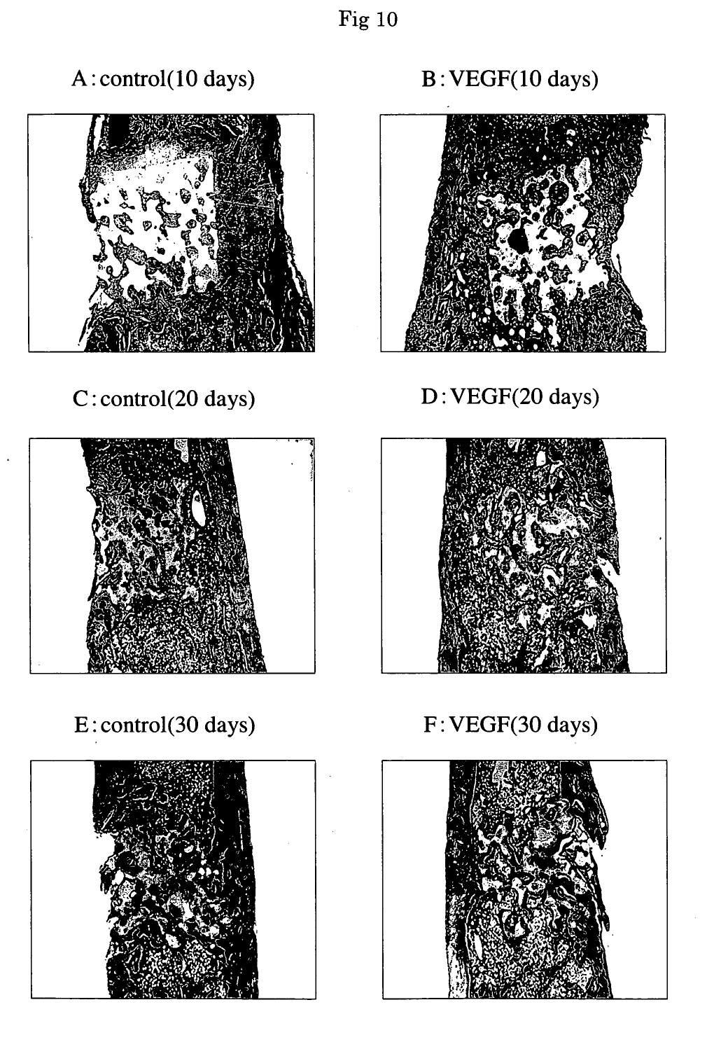

The results are shown in FIG. 10. “cont1” and “cont2” independently represent a group uninfected with viruses (controls). “cont1” is a low-magnification image, and “cont2” is a high-magnification image. VEGF1 and VEGF2 are independently a group infected with viruses. VEGF1 is a low-magnification image, and VEGF2 is a high-magnification image. As is apparent from FIG. 10, osteogenesis did not take place to a significant extent in the uninfected group. However, significant osteogenesis was clearly observed in the infected group.

All publications, patents, and patent applications cited herein are incorporated herein by reference in their entirety.

### INDUSTRIAL APPLICABILITY

The present invention enables cells to rapidly differentiate and proliferate into target tissues, thereby effectively regenerating bones. Thus, the present invention can provide excellent alternative bone implants in the field of regenerative medicine.

1. Alternative bone implants consisting of a bioadaptable material that comprises cells transfected with vascular endothelial growth factor (VEGF) genes.

2. (canceled)

3. (canceled)

4. The implants according to claim 1, wherein the cells are embryonic stem cells or bone-marrow-derived mesenchymal stem cells.

5. The implants according to claim 4, wherein the cells are osteoblasts.

6. The implants according to claim 5, wherein the cells are harvested from a patient.

7. The implants according to claim 1, wherein the bioadaptable material is selected from the group consisting of hydroxyapatite, A-TCP, β-TCP, collagen, polylactic acid, polyglycolic acid, and a complex of two or more thereof.

8. A method for producing alternative bone implants comprising the following procedures of:
1) inducing differentiation of bone-marrow-derived cells into osteoblasts in vitro;
2) transfecting vascular endothelial growth factor (VEGF) genes into the cells; and
3) inoculating the cells into bioadaptable material for proliferation.

9. (canceled)

10. The method according to claim 8, wherein the bioadaptable material is selected from the group consisting of hydroxyapatite, α-TCP, β-TCP, collagen, polylactic acid, polyglycolic acid, and a complex of two or more thereof.

11. The method according to claim 8, wherein the growth factor VEGF genes are transfected using an adenoviral or retroviral vector.

12. The method according to claim 8, wherein differentiation is induced with the aid of a member selected from the group consisting of dexamethasone, immunosuppressants, bone morphogenetic proteins, and osteogenic humoral factors.

13. A method of bone regeneration comprising the following procedures of:
1) inducing differentiation of bone-marrow-derived cells into osteoblasts in vitro;
2) transfecting vascular endothelial growth factor (VEGF) genes into the cells;
3) inoculating the cells into bioadaptable material for proliferation; and
4) transplanting the bioadaptable material with the cells and/or tissues regenerated from the cells into a patient.

14. The method according to claim 13, wherein the bioadaptable material is selected from the group consisting of hydroxyapatite, α-TCP, β-TCP, collagen, polylactic acid, polyglycolic acid, and a complex of two or more thereof.

15. The method according to claim 13, wherein the VEGF genes are transfected using an adenoviral or retroviral vector.

16. The method according to claim 13, wherein differentiation is induced with the aid of a member selected from the group consisting of dexamethasone, immunosuppressants, bone morphogenetic proteins, and osteogenic humoral factors.
